(19) United States
(12) Patent Application Publication
(10) Pub. No.: US 2025/0366895 A1
(43) Pub. Date: Dec. 4, 2025

(54) LONG LOCKING ATTACHMENT WASHER FOR NAIL-PLATE COMBO CONSTRUCT

(71) Applicant: DePuy Synthes Products, Inc., Raynham, MA (US)

(72) Inventor: Kenny Koay, West Chester, PA (US)

(73) Assignee: DePuy Synthes Products, Inc., Raynham, MA (US)

(21) Appl. No.: 19/223,669

(22) Filed: May 30, 2025

Related U.S. Application Data
(60) Provisional application No. 63/654,458, filed on May 31, 2024.

(51) Int. Cl. A61B 17/80; A61B 17/86

(52) U.S. Cl. CPC A61B 17/80; A61B 17/86; A61B 2017/8655

(57) ABSTRACT

According to an embodiment of the present disclosure, a bone fixation system includes an intramedullary nail and a bone plate. The plate body can be subdivided into different regions, including but not limited to a mid-shaft region and a proximal shaft region. The mid-shaft region and the proximal shaft region optionally have different widths and/or thicknesses in order to increase flexibility of the bone plate. The bone plate defines one or more plate fixation holes, which may be bone fixation holes. The plate fixation holes extend from the outer surface to the bone-facing surface, and are configured to receive various fixation members for affixing the bone plate to the IM nail and/or to the bone. One or more of the plate fixation holes are connected to the bone plate via protruding tabs, such that the plate fixation holes are separate from, yet connected to, the bone plate.

### CROSS-REFERENCE TO RELATED APPLICATIONS

This application claims the benefit of U.S. Provisional Patent Application No. 63/654,458, filed on May 31, 2024, the entire contents of which are incorporated herein by reference.

### TECHNICAL FIELD

The present invention relates to bone plates, and more particularly to bone plates having variable thickness and/or width along their length, as well as bone plates having regions with one or more protruding tabs.

### BACKGROUND

Long-bone fractures, such as femoral and humeral fractures, are often treated with screws or other fixation devices inserted into, or through, a bone to stabilize fractured portions thereof once they have been brought into corrective alignment. Femoral bone fixation treatments can involve the insertion of an intramedullary (IM) nail into the medullary cavity of the femur and a subsequent insertion of bone fixation screw(s) into a condylar or trochanteric portion of the femur, depending upon whether the IM nail is inserted into the medullary canal at an antegrade or retrograde insertion trajectory. Antegrade insertion trajectories extend from the anatomical proximal end of the femur (e.g., at the hip joint), such as from the tip or slightly lateral to the tip of the greater trochanter, and into the medullary canal toward the anatomical distal end of the femur along the anatomical axis of the femur. Retrograde insertion trajectories extend from the anatomical distal end of the femur (e.g., at the knee joint) toward the anatomical proximal end of the femur and are effectively the opposite of antegrade insertion trajectories.

As used herein, the term “retrograde intramedullary nail” refers to an IM nail designed for retrograde insertion into the medullary canal. Retrograde IM nails are known to provide advantageous fixation to the distal portions of the femur (e.g., the distal condylar and intercondylar regions), such as for treating distal femur fractures. For example, retrograde IM nails allow for easier targeting and insertion of locking screws within locking holes at the trailing end of the nail, which resides within the distal femur. When treating distal femur fractures, retrograde femoral nailing can require additional bone fixation, particularly when the femur exhibits poor bone quality and/or in periprosthetic settings. In such instances, a supplemental locking attachment washer (LAW) or plate can be coupled to the trailing end of the nail via a plurality of bone screws. For simplicity and brevity, the locking attachment washers described below can each be referred to as a “plate.” The plate defines one or more holes for bone screws that interconnect with the IM nail. Additionally, the plate can define one or more additional holes for additional bone screws that affix within portions of the femur adjacent the trailing end of the IM nail for supplemental bone fixation.

IM nails and associated plates can be provided at various sizes, geometries, and lengths to facilitate treatments of various types and indications of bone fractures. Additionally, physicians can select various IM nail and plate combinations based upon the treatment needs of the patient. For example, IM nails can be paired with plates having an increased length, such that the plate can extend from an end region of the bone (e.g., the condylar or trochanteric portion of the femur) to and alongside the shaft region of the bone. For such plates having increased length, the distal end of the plate should be fixed to the bone in alignment with the associated lengthwise portion of the IM nail.

The targeting and insertion of locking screws at the trailing end of the IM nail, and at the associated plate, is generally simplified by the fact that the nail trailing end and/or the associated plate can be directly engaged with instrumentation, such as an insertion handle and/or an aiming arm having aiming elements for targeting the locking holes near the trailing end of the IM nail. However, targeting locking holes at the leading end of an IM nail, however, is more challenging due to factors such as nail deflection and deformation that can result from stress and strain, particularly as the length of the nail increases. Furthermore, as the length of the plate increases, the challenges relating to aligning the distal end of the plate with the associated portion of the IM nail also increase.

### SUMMARY

According to an embodiment of the present disclosure, a bone fixation system includes an intramedullary nail and a bone plate. The intramedullary nail has a nail body elongate along a longitudinal direction. The bone plate has a plate body extending along a longitudinal plate axis and having first and second sides opposite each other along a lateral direction perpendicular to the longitudinal plate axis. The plate body has an outer surface and a bone-facing surface opposite each other. The plate body is alignable with the nail body such that: the longitudinal plate axis is oriented substantially along the longitudinal direction, the outer surface and bone-facing surface are spaced from each other along the transverse direction, and the lateral direction is offset from the longitudinal and transverse directions. The plate body can be subdivided into different regions, including but not limited to a mid-shaft region and a proximal shaft region. The mid-shaft region and the proximal shaft region optionally have different widths and/or thicknesses in order to increase flexibility of the bone plate.

The bone plate defines one or more plate fixation holes, which may be bone fixation holes. The plate fixation holes extend from the outer surface to the bone-facing surface, and are configured to receive various fixation members for affixing the bone plate to the IM nail and/or to the bone. According to an embodiment of the present disclosure, one or more of the plate fixation holes are connected to the bone plate via protruding tabs, such that the plate fixation holes are separate from, yet connected to, the bone plate.

### BRIEF DESCRIPTION OF THE DRAWINGS

The foregoing summary, as well as the following detailed description of illustrative embodiments of the present application, will be better understood when read in conjunction with the appended drawings. For the purposes of illustrating the features of the present application, there is shown in the drawings illustrative embodiments. It should be understood, however, that the application is not limited to the precise arrangements and instrumentalities shown. In the drawings:

FIGS. 1A-1C are lateral views of a bone fixation system including a nail-plate construct that includes an IM nail interconnected with a bone plate, according to an embodiment of the present disclosure; and

FIGS. 2A-2C are anterior-posterior views of a bone fixation system including a nail-plate construct that includes an IM nail interconnected with a bone plate, according to an embodiment of the present disclosure.

### DETAILED DESCRIPTION OF ILLUSTRATIVE EMBODIMENTS

The present disclosure can be understood more readily by reference to the following detailed description taken in connection with the accompanying figures and examples, which form a part of this disclosure. It is to be understood that this disclosure is not limited to the specific devices, methods, applications, conditions or parameters described and/or shown herein, and that the terminology used herein is for the purpose of describing particular embodiments by way of example only and is not intended to be limiting of the scope of the present disclosure. Also, as used in the specification including the appended claims, the singular forms “a,” “an,” and “the” include the plural, and reference to a particular numerical value includes at least that particular value, unless the context clearly dictates otherwise.

The term “plurality”, as used herein, means more than one. When a range of values is expressed, another embodiment includes from the one particular value to the other particular value. Similarly, when values are expressed as approximations, by use of the antecedent “about,” it will be understood that the particular value forms another embodiment. All ranges are inclusive and combinable.

The terms “approximately”, “about”, and “substantially”, as used herein with respect to dimensions, angles, ratios, and other geometries, take into account manufacturing tolerances. Further, the terms “approximately”, “about”, and “substantially” can include 10% greater than or less than the stated dimension, ratio, or angle. Further, the terms “approximately”, “about”, and “substantially” can equally apply to the specific value stated.

It should be understood that, although the terms first, second, etc. may be used herein with reference to various features, these features should not be limited by these terms other than to distinguish one feature from another. For example, a first feature could be termed a second feature in another context, and, similarly, a second feature could be termed a first feature in another context, without departing from the scope of the embodiments disclosed herein.

The embodiments disclosed herein pertain to bone plates that are adapted to increase flexibility. The example embodiments described below include bone fixation systems that include an intramedullary (IM) nail and a bone plate having a hole structure that allows the bone plate to interconnect with the IM nail. The nail length can be in a range of about 120 mm to about 500 mm. The nail width can be in a range of about 8 mm to about 16 mm. The plate length can be in a range of about 120 mm to about 550 mm.

The bone plate may be subdivided into regions with different lengths, widths, and/or thicknesses. For example, the bone plate may be subdivided into a plate head region, a mid-shaft region, and/or a proximal shaft region. The plate head region and/or the proximal shaft region may comprise one or more plate holes (e.g., which may be referred to as plate fixation holes, nail holes, locking holes, variable angle locking (VAL) holes, etc.) that may receive respective locking members (e.g., nails, screws, VAL screws, etc.) for locking the bone plate to a bone and/or an intramedullary (IM) nail. The mid-shaft region may be differentiated from the plate head region and the proximal shaft region by its lack of plate holes. The mid-shaft region and the proximal shaft region may have differing widths, lengths, and/or thicknesses (e.g., differing stiffness and/or bending strengths), which may increase flexibility of the bone plate, and may allow for increased customization. Additionally, increasing flexibility of the bone plate may prevent the bone plate from breaking when flexed beyond a threshold limit.

Additionally, the plate head region may comprise a main body and one or more protruding tabs. The main body and the protruding tabs may each comprise one or more plate holes for receiving respective locking members for locking the bone plate to the bone and/or IM nail. The protruding tabs may be separated from the main body of the plate head region, but may be connected to the main body by one or more screw hole tabs (e.g., relatively thin attachments), which may allow in-situ bending adapting to patient anatomy to minimize soft tissue irritation.

An exemplary embodiment of a bone fixation system includes an intramedullary (IM) nail insertable within the medullary canal of a long bone, a bone plate affixable to an outer surface of the bone and connectable to the IM nail, and a plurality of fixation members for coupling the bone plate to the IM nail (e.g., thereby forming an interconnected nail-plate construct (NPC)) and/or for affixing the nail-plate construct to the bone. The NPC can also be configured to treat periprosthetic conditions, including for affixing with an existing implant or prosthesis. It should be appreciated that the foregoing represents one non-limiting example of a particular treatment that the NPC can provide. Additionally, although the long bone discussed with reference to the illustrated embodiments is a femur, it should be appreciated that the bone fixation system can be adapted for use with other long bones, such as a tibia, fibula, humerus, radius, and/or ulna, by way of non-limiting examples. Additionally, although the IM nail shown in the illustrated embodiments is configured for retrograde femoral insertion, the bone fixation system can be adapted for use with antegrade insertion trajectories.

The IM nail has a first end and a second end spaced from each other along a longitudinal direction. In the illustrated embodiment, which shows a retrograde femoral IM nail, the longitudinal direction generally extends along the cranial-caudal direction of patient anatomy. In such embodiments, the first end is the proximal end of the nail and is configured to temporarily couple with insertion instrumentation, such as an insertion arm. The second end is the distal end of the nail and is the leading or forward end of the nail during insertion within the medullary canal. The second end is spaced from the first end in a distal direction, while the first end is spaced from the second end is a proximal direction that is opposite the distal direction. It should be appreciated that the distal and proximal directions are each mono-directional components of the longitudinal direction, which is bi-directional. The IM nail defines a nail length measured between the first and second ends along the longitudinal direction. The IM nail also defines a nail width measured along a direction perpendicular to the longitudinal direction. The bone fixation system can include IM nails of various lengths or widths for selective use for various types of bone fractures, as described below.

It should also be appreciated that, as used herein: the terms “longitudinal”, “longitudinally”, and derivatives thereof refer to the longitudinal direction; the terms “distal”, “distally”, and derivatives thereof refer to the distal direction; and the terms “proximal”, “proximally”, and derivatives thereof refer to the proximal direction. Furthermore, because retrograde insertion trajectories essentially place the structural (e.g., implant) uses of the directional terms “proximal” and “distal” into opposition with these terms' anatomical usage (e.g., the “proximal” end of the IM nail resides in the “distal” femur, while the “distal” end of the nail is spaced toward the “proximal” femur) directions, for purposes of clarity, the terms “proximal” and “distal” and their derivatives are used herein to refer to directional aspects of the synthetic structural components of the bone fixation system, unless, however, these terms are used with specific reference to anatomy (e.g., the “proximal femur, “distal femur”, “distal anatomical direction”), in which latter case the terms refer to the anatomical directions.

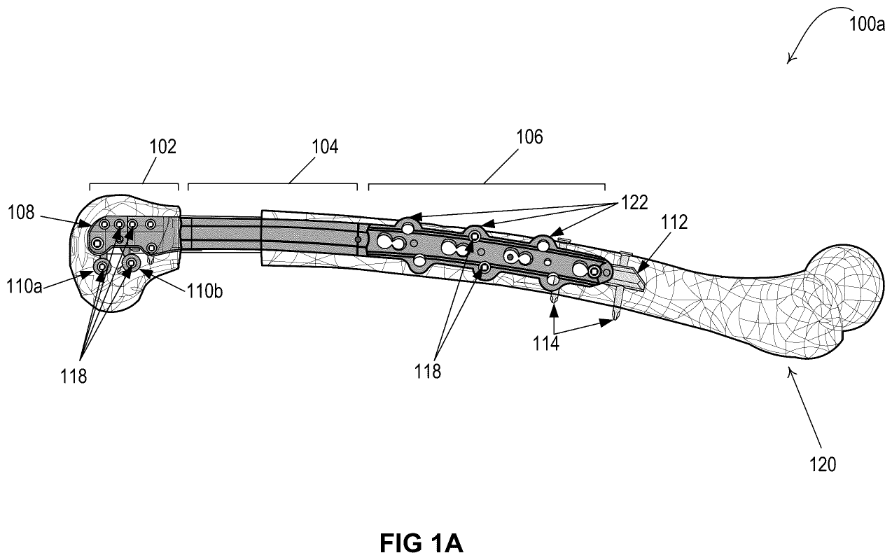

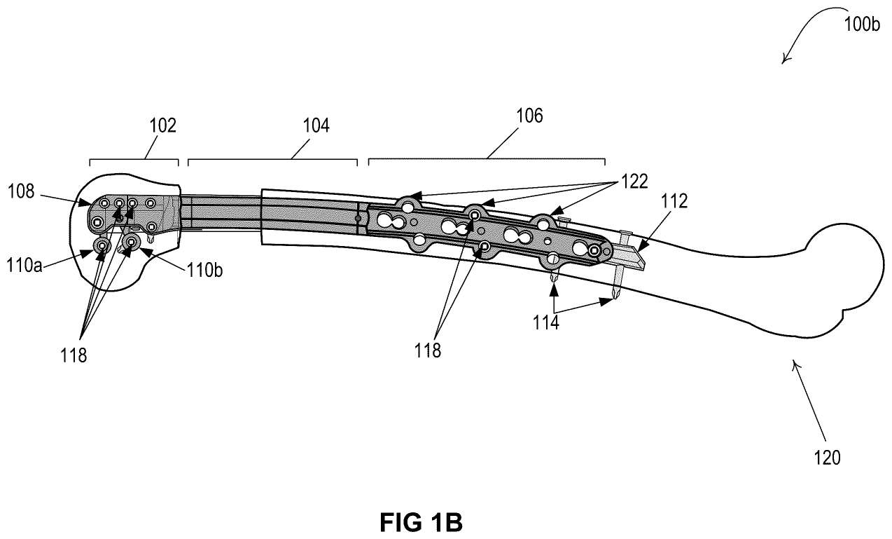

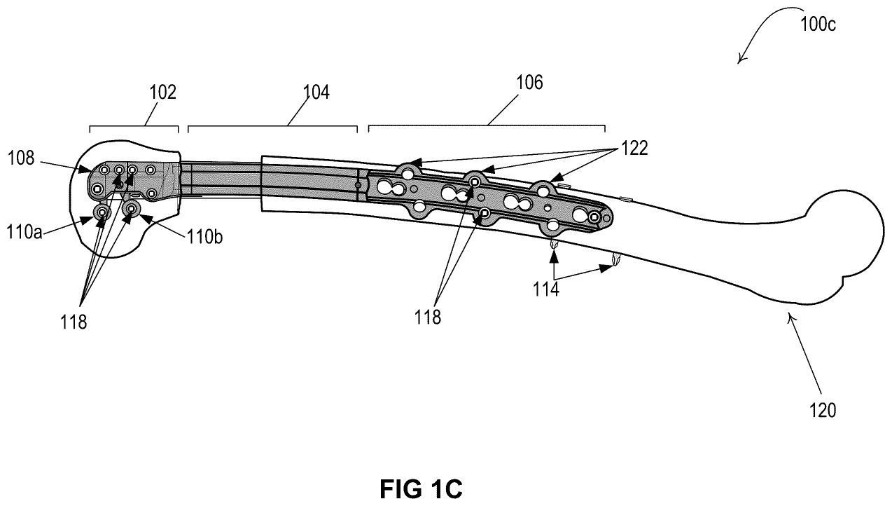

FIGS. 1A-1C illustrate respective lateral views 100a, 100b, and 100c of a bone fixation system including a nail-plate construct that includes an IM nail 112 interconnected with a bone plate according to an embodiment of the present disclosure. The IM nail 112 has a nail body that extends along a central nail axis that is generally oriented along the longitudinal direction. The central nail axis need not be linear; preferably, the central nail axis follows a path that is substantially coextensive with an anatomical axis of a bone 120. Thus, the central nail axis can have one or more straight portions and/or one or more curved portions. The nail body includes a proximal locking portion (also referred to herein as the “nail head”) that extends distally from a first end, an intermediate portion that extends distally from the nail head, and a distal locking portion that extends from the intermediate portion to a second end of the nail 112. The nail head is configured to attach to instrumentation, such as an insertion handle, for inserting the nail 112 into the medullary canal.

As shown in FIGS. 1A-1C, the bone plate may include a plate body, which may be subdivided into a plate head region 102, a mid-shaft region 104, and/or a proximal shaft region 106. The plate body may extend along a longitudinal plate axis. Although the long bone shown in FIGS. 1A-1C is a femur, it should be appreciated that the bone fixation system can be adapted for use with other long bones, such as a tibia, fibula, humerus, radius, and/or ulna, by way of non-limiting examples.

According to an embodiment of the present disclosure, the mid-shaft region 104 and the proximal shaft region 106 may be made of the same material, but may have different widths and/or thicknesses. The width and/or thickness (e.g., stiffness and/or bending strength) of the mid-shaft region 104 may be variable, and may be less than the width and/or thickness of the proximal shaft region. For example, a ratio of the width of the mid-shaft region 104 to the width of the proximal shaft region 106 may be within a range of approximately 0.55 to 1, and more preferably within a range of approximately 0.7 to 0.9. Similarly, a ratio of the thickness of the mid-shaft region 104 to the thickness of the proximal shaft region 106 may be within a range of approximately 0.33 to 1, and more preferably within a range of approximately 0.7 to 0.9. For example, the thickness of the mid-shaft region 104 may be approximately 3 mm, while the thickness of the proximal shaft region may be approximately 4.2 mm, leading to a ratio of approximately 0.715. In an example, the thickness of the mid-shaft region 104 may be approximately within a range of 2 mm to 6 mm. For example, the thickness of the mid-shaft region 104 may be approximately 2.3 mm, 3 mm, 3.5 mm, or 4.5 mm, inclusive. The thickness of the proximal shaft region 106 may be approximately within a range of 4.2 mm to 6 mm. The width of the mid-shaft region 104 may be approximately within a range of 10 mm to 17.5 mm. The width of the proximal shaft region 106 may be approximately 17.5 mm. Decreasing the width and/or thickness of the mid-shaft region 104 relative to the proximal shaft region 106 and/or the plate head region 102 may decrease the stiffness of the bone plate, which may thereby increase flexibility of the bone plate. Increasing flexibility of the bone plate may prevent the bone plate from breaking when flexed, but may instead allow the bone plate to bend when flexed.

Additionally, the mid-shaft region 104 and the proximal shaft region 106 may have respective bending strengths, which may be a property of the width, thickness, and or type of material used. The respective bending strengths may indicate respective flexibilities of the mid-shaft region 104 and the proximal shaft region 106, and may further indicate respective maximum stress that the mid-shaft region 104 and the proximal shaft region 106 can withstand before yielding in a bending test. A ratio of the bending strength of the mid-shaft region 104 to the bending strength of the proximal shaft region 106 may be approximately within a range of 0.2 to 1, and more preferably within a range of approximately 0.7 to 0.9. In an example, the bending strength of the proximal shaft region 106 may be defined as 100, and the bending strength of the mid-shaft region 104 may therefore be within a range of 20 to 100 when measured in the same units.

As shown in FIGS. 1A-1C, the plate head region 102 and/or the proximal shaft region 106 may comprise one or more plate holes 118 that may receive respective locking members (e.g., nails, screws, VAL screws, etc.) for locking the bone plate to the bone 120 and/or IM nail 112. The IM nail 112 may be inserted into a medullary canal of the bone 120 during a surgical procedure. The plate holes 118 may be, for example, plate fixation holes that extend from the outer surface of the bone plate to the bone-facing surface of the bone plate. The plate fixation holes 118 are configured to receive various fixation members for affixing the bone plate to the IM nail 112 and/or to the bone 120. For example, the locking members may be VAL screws, nails, standard locking screws, condylar nut and washer screws, locking attachment washers (LAWs), low profile locking screws, and/or any other type of locking members. The plate head region 102 and/or the proximal shaft region 106 may have approximate widths and/or thicknesses to accommodate the locking members. For example, the proximal shaft region 106 may have an appropriate thickness and/or width to accommodate 3.5 mm and/or 5.0 mm VAL holes configured to receive one or more VAL screws, as well as other plate fixation holes configured to receive other types of nails and/or screws. The placement of the plate holes 118 on the plate body may be located such that they are able to be connected to the IM nail 112 and/or the bone 120.

As shown in FIGS. 1A-1C, the mid-shaft region 104 may be distinguished from the plate head region 102 and/or the proximal shaft region 106 in that the mid-shaft region 104 may not have any plate holes (e.g., the mid-shaft region 104 may lack plate holes). For example, the mid-shaft region may comprise a solid body.

Further, as shown in FIGS. 1A-1C, the plate head region 102 may comprise a main body 108 and one or more protruding tabs 110a, 110b. The protruding tabs 110a, 110b may extend from the main body 108, and may be connected to the main body 108 by one or more screw hole tabs. The protruding tabs may be separated from the main body of the plate head region, but may be connected to the main body by one or more screw hole tabs, which may reduce the overall weight and size of the bone plate. The protruding tabs 110a, 110b may be connected to a lower end of the plate head region 102, such that the protruding tabs 110a, 110b are part of the plate head region 102 and not the mid-shaft region 104. Each of the protruding tabs 110a, 110b may define a respective plate fixation hole 118 that is configured to receive a respective fixation member for affixing the bone plate to the IM nail 112 and/or to the bone 120. Further, the main body 108 of the plate head region 102 may define one or more plate fixation holes 118. Additionally, one or more fixation members 114 may be connected to the IM nail 112 but not the plate body of the bone plate.

As shown in FIGS. 1A-1C, the proximal shaft region 106 may have protrusions 122 from the main plate body. Each protrusion may define a respective plate fixation hole 118 that is configured to receive a respective fixation member for affixing the bone plate to the IM nail 112 and/or to the bone 120. The protrusions 122 may extend from the sides of the proximal shaft region 106 such that the overall width of the proximal shaft region 106 is increased. Additionally, the mid-shaft region 104 may not have any protrusions, which may further reduce the width of the mid-shaft region 104 relative to the proximal shaft region 106.

The IM nail 112 and the bone plate can be provided at various configurations and sizes adapted for treating various conditions. For example, the IM nail 112 and bone plate can be tailored as needed to form various nail-plate constructs adapted for treating supracondylar fractures (including those with intra-articular extension), combinations of ipsilateral condylar and diaphyseal fractures, ipsilateral femoral/tibial fractures, femoral fractures in multiple-trauma patients, periprosthetic fractures (e.g., including Type B1 and Type C according to the Vancouver classification system, and also including inter-prosthetic fractures), fractures in morbidly obese patients, fractures in osteoporotic and osteopenic bone, impending pathological fractures, malunions, and non-unions, by way of non-limiting examples. It should be appreciated that the nail-plate construct can be adapted as needed to treat various other conditions. Additionally, as described above, different portions of the IM nail 112 and/or the bone plate may have different lengths, thicknesses, and/or widths.

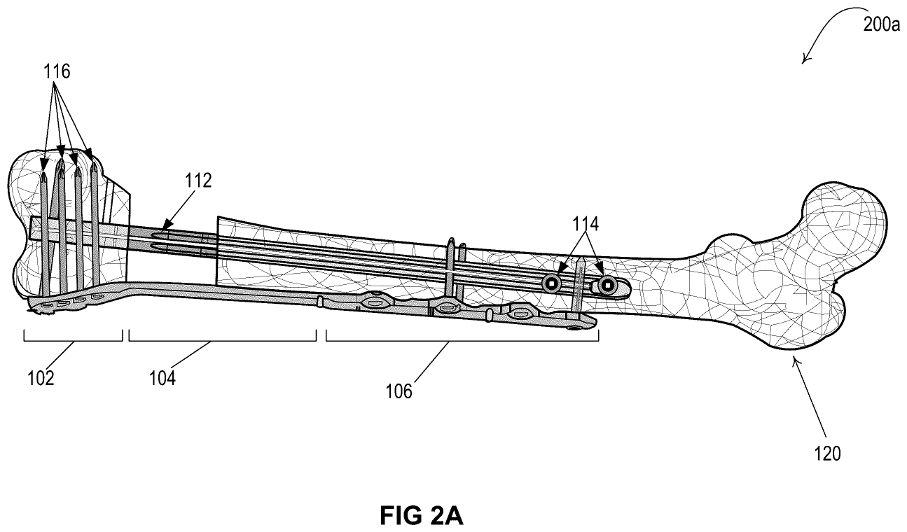

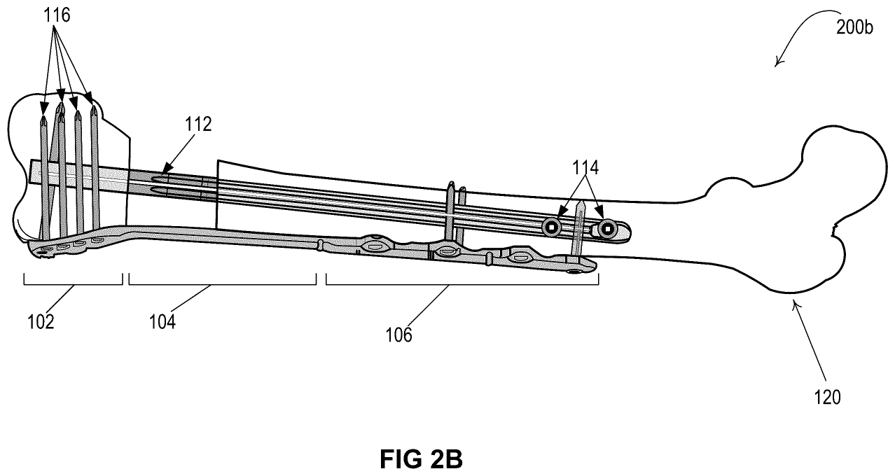

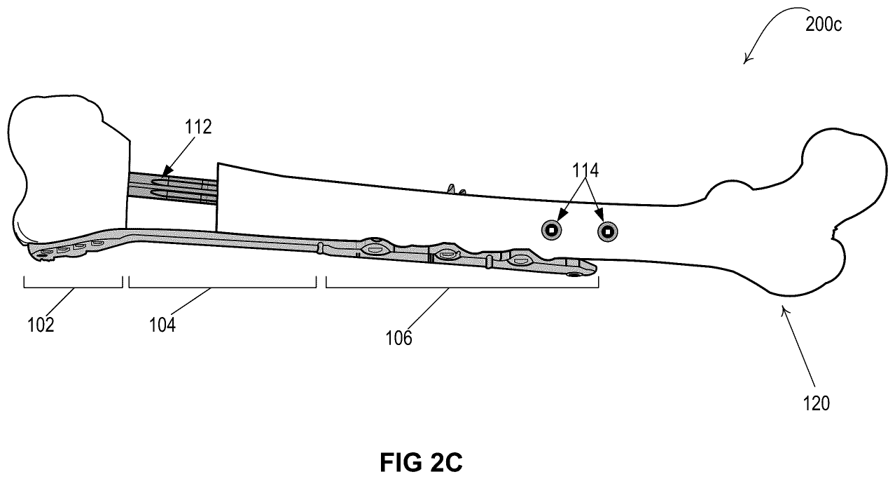

FIGS. 2A-2C illustrate respective anterior-posterior views 200a, 200b, 200c of a bone fixation system including a nail-plate construct that includes an IM nail 112 interconnected with a bone plate, according to an embodiment of the present disclosure. For example, the nail-plate construct shown in FIGS. 2A-2C may include the bone plate including the plate body, which may be defined by the plate head region 102, the mid-shaft region 104, and the proximal shaft region 106, as well as the IM nail 112, the locking members 114, and the bone 120 described with reference to FIGS. 1A-1C. Although the long bone shown in FIGS. 2A-2C is a femur, it should be appreciated that the bone fixation system can be adapted for use with other long bones, such as a tibia, fibula, humerus, radius, and/or ulna, by way of non-limiting examples.

Referring again to FIGS. 2A-2C, there may be locking members 116 that connect the plate body (e.g., the plate head region 102) to the bone 120 and/or the IM nail 112. For example, each of the locking members 116 may be inserted into a respective plate fixation hole of the plate head region 102, which be one of the plate fixation holes 118 of the main body 108 or of one of the protruding tabs 110a, 110b shown in FIGS. 1A-1C. One or more of the locking members 116 may be VAL screw(s) that connect the plate body to the IM nail, while the other locking members 116 may be screws that connect the plate body to the bone 120. The plate fixation hole that receives the VAL screw may be referred to as a VAL hole.

It should be appreciated that the bone plate described above can be employed to treat a bone without the use of an accompanying IM nail. In such embodiments, a hole arrangement can be employed to facilitate plate angulation (e.g., pivoting about a first locking member partially inserted through the first hole and into underlying bone) for proper plate alignment with underlying bone, in similar fashion as described above. In yet further embodiments, the bone plate described above can be employed with other types of implants, such as a second bone plate on an opposite side of a bone, for facilitating angulation of the bone plate about a first locking member partially inserted through a first hole and into a hole or other structure of the second bone plate.

Although the disclosure has been described in detail, it should be understood that various changes, substitutions, and alterations can be made herein without departing from the spirit and scope of the invention as defined by the appended claims. Moreover, the scope of the present disclosure is not intended to be limited to the particular embodiments described in the specification. In particular, one or more of the features from the foregoing embodiments can be employed in other embodiments herein. As one of ordinary skill in the art will readily appreciate from that processes, machines, manufacture, composition of matter, means, methods, or steps, presently existing or later to be developed that perform substantially the same function or achieve substantially the same result as the corresponding embodiments described herein may be utilized according to the present disclosure.

What is claimed is:

1. A bone plate comprising:
a plate body extending along a longitudinal plate axis, wherein the plate body comprises a plate head region, a mid-shaft region, and a proximal shaft region, the mid-shaft region and the proximal shaft region having respective widths and thicknesses;
wherein the plate head region comprises a main body and one or more protruding tabs extending from the main body, each of the one or more protruding tabs defining respective plate holes configured to receive respective locking members for insertion for interconnecting the bone plate to one or more of a bone or an intramedullary nail; and
wherein at least one of the width or thickness of the mid-shaft region is different from the width or thickness of the proximal shaft region, respectively.

2. The bone plate of claim 1, wherein a ratio of the width of the mid-shaft region to the width of the proximal shaft region is within a range of 0.55 to 1.

3. The bone plate of claim 1, wherein a ratio of the width of the mid-shaft region to the width of the proximal shaft region is within a range of 0.7 to 0.9.

4. The bone plate of claim 1, wherein a ratio of the thickness of the mid-shaft region to the thickness of the proximal shaft region is within a range of 0.33 to 1.

5. The bone plate of claim 1, wherein a ratio of the thickness of the mid-shaft region to the thickness of the proximal shaft region is within a range of 0.7 to 0.9.

6. The bone plate of claim 1, wherein the proximal shaft region comprises one or more respective plate holes configured to receive respective locking members for insertion for interconnecting the bone plate to one or more of the bone or the intramedullary nail.

7. The bone plate of claim 6, wherein the one or more respective plate holes comprised in the proximal shaft region comprise a variable angle locking (VAL) hole configured to receive a VAL screw configured for variable angle locking.

8. The bone plate of claim 1, wherein the mid-shaft region comprises a solid body.

9. The bone plate of claim 1, wherein the main body of the plate head region further comprises one or more respective plate holes configured to receive respective locking members for insertion for interconnecting the bone plate to one or more of the bone or the intramedullary nail.

10. The bone plate of claim 9, wherein the one or more respective plate holes comprised in the main body of the plate head region comprise a VAL hole configured to receive a VAL screw configured for variable angle locking.

11. The bone plate of claim 1, wherein the mid-shaft region and the proximal shaft region have respective bending strengths, and wherein a ratio of the bending strength of the mid-shaft region to the bending strength of the proximal shaft region is within a range of 0.2 to 1.

12. The bone plate of claim 1, wherein the mid-shaft region and the proximal shaft region have respective bending strengths, and wherein a ratio of the bending strength of the mid-shaft region to the bending strength of the proximal shaft region is within a range of 0.7 to 0.9.

13. A bone plate comprising:
a plate body extending along a longitudinal plate axis, wherein the plate body comprises a plate head region, a mid-shaft region, and a proximal shaft region, the mid-shaft region and the proximal shaft region having respective widths and thicknesses;
wherein at least one of the width or thickness of the mid-shaft region is different from the width or thickness of the proximal shaft region, respectively.

14. The bone plate of claim 13, wherein a ratio of the width of the mid-shaft region to the width of the proximal shaft region is within a range of 0.7 to 0.9.

15. The bone plate of claim 13, wherein a ratio of the thickness of the mid-shaft region to the thickness of the proximal shaft region is within a range of 0.7 to 0.9.

16. The bone plate of claim 13, wherein the mid-shaft region and the proximal shaft region have respective bending strengths, and wherein a ratio of the bending strength of the mid-shaft region to the bending strength of the proximal shaft region is within a range of 0.7 to 0.9.

17. A bone plate comprising:
a plate body extending along a longitudinal plate axis, wherein the plate body comprises a plate head region, a mid-shaft region, and a proximal shaft region;
wherein the plate head region comprises a main body and one or more protruding tabs extending from the main body, each of the one or more protruding tabs defining respective plate holes configured to receive respective locking members for insertion for interconnecting the bone plate to one or more of a bone or an intramedullary nail.

18. The bone plate of claim 17, wherein the one or more protruding tabs are connected to the main body by respective screw hole tabs.

19. The bone plate of claim 17, wherein the protruding tabs are connected to a lower end of the plate head region.
